(19) United States
(12) Patent Application Publication
(10) Pub. No.: US 2009/0176738 A1
(43) Pub. Date: Jul. 9, 2009

(54) Dermal medicament delivery system

(76) Inventors: Leonard Mackles, New York, NY (US); William Bess, Edison, NJ (US)

Correspondence Address: OMRI M. BEHR, 325 PIERSON AVENUE, EDISON, NJ, 08837-3123, US

(21) Appl. No.: 12/316,003

(22) Filed: Dec. 9, 2008

Related U.S. Application Data
(60) Provisional application No. 61/019,649, filed on Jan. 8, 2008.

(51) Int. Cl. A61K 31/695; A61K 31/135; A61P 17/00; A61K 31/216

(52) U.S. Cl. 514/63; 514/648; 514/532

(57) ABSTRACT

There is provided a means of applying a heated medicament to the skin and concurrently hydrating the skin thus raising the efficacy of administration.

### RELATED APPLICATIONS

This application claims priority of Provisional Application 61/01638 filed Dec. 26, 2007, Provisional Application 61/019649 filed Jan. 14, 2008 and is a continuation in part of CHAV3.0-046 (SN unallocated) filed Dec. 04, 2008.

### FIELD OF THE INVENTION

This invention relates to a method for increasing the efficacy of medicaments delivered to the skin of mammals.

### BACKGROUND

It is well known in the art that the efficacy of topically delivered medicaments can be improved by warming the medicament and/or skin just prior to application. It is also well known in the art that the efficacy can be improved by insuring that the skin is well hydrated just prior to application of the medicament.

### SUMMARY OF THE INVENTION

The present invention provides a means of applying a heated medicament to the skin and concurrently hydrating the skin thus raising the efficacy of administration. In the present invention there is provided a dual phase aqueous based system which self-heats when the two phases are combined. The medicament(s) can be included in either or both phases. Self-warming is achieved through the use of an oxidation-reduction reaction alone or in combination thereof with an acid-base reaction.

The invention provides an improved method of applying at least one dermally or transdermally physiologically active medicament to the skin of a mammal in need of same consisting essentially of a first dermally acceptable phase and a second dermally acceptable phase mixable therewith, wherein at least one of said phases has an aqueous component.

The medicament may be present in either phase. In one embodiment, the first phase consists essentially of at least one component capable of producing an exothermic reaction of dermally acceptable magnitude when contacted with the second phase and promptly applying the mixture to the skin of the subject.

The first phase may be a reducing phase and the second phase is then an oxidizing phase. Alternatively where first phase is a basic phase, the second phase is an acidic phase. The invention however is not so limited. The first phase, when reducing, may additionally comprises a basic component and said second phase when acid will comprise an acidic component reactable with said basic component. Similarly first reducing phase may additionally comprise an acidic component and then the second phase comprises a basic component reactable with said acidic component.

The invention also provides a kit for applying at least one dermally or transdermally physiologically active medicament to the skin of a mammal in need of same, consisting essentially of a first dermally acceptable phase and a separate second dermally acceptable phase mixable therewith.

The medicament may be present in either phase. The first phase consists essentially of at least one component capable of producing an exothermic reaction of dermally acceptable magnitude when contacted with said second phase and wherein at least one of said phases has an aqueous component.

In the kit, where the first phase is a reducing phase the second phase is an oxidizing phase. Alternatively, where the first phase is a basic phase, the said second phase is an acidic phase.

Where the first phase is a reductive phase it may additionally comprise a basic component, then the said second, oxidative phase comprises an acidic component reactable with said basic component. Similarly when the first reductive phase additionally comprises an acidic component and the second phase oxidative comprises a basic component reactable with said acidic component.

When the two phases are combined, the reaction(s) which occur, result in a dramatic increase in temperature. The increased temperature combined with the aqueous vehicle hydrates the skin and heats the medicament(s). The result is improved product efficacy.

### Medicaments

Acceptable medicaments include any drug suitable for topical delivery, whether intended for systemic treatment via transdermal delivery or intended to treat a topical skin condition. Non-limiting examples include analgesic drugs, analgesic anti-inflammatory drugs, central nervous system drugs, antihistaminic or antiallergic drugs, acitonide anti-inflammatory drugs, androgenic and estrogenic steroids, -respiratory drugs, sympathomimetic drugs, antimicrobial drugs, antihypertensive drugs, cardiotonic drugs, coronary vasodilators, vasoconstrictors, beta blocking and antiarrhythemic drugs, calcium antagonistic and other circulatory anticonvulsants, anti-vertigo-tranquilizing drugs, antipsychotic drugs, muscle-reactants drugs, anti-Parkinson drugs, non-steroidal hormones, anti-hormones, vitamins, antitumor, enzymes, herb medicines or crude extracts, miotics, cholinergic agonists, antimuscarinic or muscarinic cholinergic blocking drugs, mydriatics, psychic energizers, humoral agents, antispasmodic drugs, antidepressants, antidiabetics, anorexic drugs, anti-allergic-drugs, decongestants, antipyretics, antimigraine drugs, antimalarial, antiulcer drugs, peptides, and anti-estrogens. Additional acceptable medicaments include cosmeceutical agents. Non-limiting examples include, moisturizers, hydroxy-acids, bleaching agents, and skin protectants.

The concentration range will vary by medicament, but it is to be understood that the acceptable range would encompass all concentrations of a particular medicament or combination of medicaments, which demonstrate acceptable efficacy.

### Heat-Producing Reactants

Heat production can be achieved by using an oxidation-reduction reaction either as the sole reaction, or in combination with an acid-base reaction. Quantities mention herein should be considered as parts by weight of the phase in which they are initially provided, unless otherwise indicated

Acceptable reducing agents include but are not limited to alkali metal or alkaline earth metal sulfites, bisulfites, thiosulfates, and metabisulfites. The acceptable range of reducing agent(s) utilized will be from 1-20%, preferably 2-15 w/w % based on total solution.

Acceptable oxidizing agents include but are not limited to peroxides. Suitably they include include hydrogen peroxide and urea peroxide. The acceptable range of peroxide(s) will depend on the concentration of reducing agent(s), and the composition of the reducing agent(s) utilized. It is to be understood that the acceptable range of peroxide will include concentrations which will result in at least a 5 degree (Fahrenheit) rise in temperature when the two phases are mixed.

Acceptable acidic reactants include but are not limited to, inorganic mineral acids, suitably sulphuric, hydrochloric, nitric and phosphoric acids preferably phosphoric acid. The range for inorganic acids is 0.05-10 w/w % preferably 0.5-5.0 w/w %, based on the aqueous diluted form selected. Similarly water soluble organic acids with an equivalent weight of less than 100, but not limited thereto, such as lactic, glycolic and citric acids may be used. In both cases the given percentages are bases on the diluted form, if present, relative to total weight of the phase.

Acceptable basic reactants include hydroxides, amines, and ammonia. The range for basic reactants will vary according to the concentration of inorganic acid used in the oxidizing phase, the particular basic reactant(s) utilized, and the final desired pH of the product. It is to be understood that the acceptable range of basic reactant(s) will encompass the range that will result in a final product pH in the range of 2-13, preferably 4-12.

### Dosage Forms

Acceptable dosage forms include any dosage form capable of delivering the medicament to a topical area. These would include liquids, lotions, gels, creams, pastes, ointments, foams, and aerosols.

### EXAMPLES

### Example 1

Lotion formulation containing diphenhydramine HCl an antihistamine, and utilizing only the oxidation-reduction reaction to produce heat.

Reducing Phase

| Ingredient | % by weight |
|---|---|
| Sodium Sulfite | 10.00 |
| Water | 67.20 |
| Hydroxypropyl Methylcellulose | 0.50 |
| Propylene Glycol | 10.00 |
| Alcohol 95% | 10.00 |
| Diazolidinyl Urea | 0.25 |
| Xanthan Gum | 1.00 |
| Diphenhydramine HCl | 1.00 |
| Total | 100.00 |

Oxidizing Phase

| Ingredient | % by weight |
|---|---|
| Hydrogen Peroxide (50%) | 5.40 |
| Water | 93.60 |
| Xanthan Gum | 1.00 |
| Total | 100.00 |

Combine approx. equal weights of each phase, mix and apply to skin. The mean rise in temperature upon mixing is ca. 50° F.

### Example 2

A lotion formulation utilizing both an oxidation-reduction reaction and an acid base reaction to produce heat. This product contains methyl salicylate, a topical anti-inflammatory medicament.

Reducing Phase

| Ingredient | % by weight |
|---|---|
| Sodium Sulfite | 8.00 |
| Sodium Metabisulfite | 2.00 |
| Monafax 785 | 4.00 |
| Span 80 | 5.00 |
| Cetyl Alcohol | 2.50 |
| Methyl Salicylate | 20.00 |
| Germaben 2 | 0.20 |
| Calcium Hydroxide | 2.00 |
| Water | 56.30 |
| Total | 100.00 |

Oxidizing Phase

| Ingredient | % by weight |
|---|---|
| Phosphoric Acid (85%) | 1.00 |
| Hydrogen Peroxide (50%) | 5.75 |
| Water | 83.25 |
| Arlacel 165 | 3.00 |
| Cetyl Alcohol | 7.00 |
| Total | 100.00 |

Combine. approx. equal weights, mix and apply to skin. The mean rise in temperature upon mixing is ca. 65° F.

### Example 3

A foam product containing a hydroxy-acid and utilizing an oxidation-reduction reaction to produce heat.

Reducing Phase

| Ingredient | % by weight |
|---|---|
| Sodium Sulfite | 12.00 |
| Lactic Acid | 3.00 |
| Lonzaine C | 10.00 |
| Glycerin | 10.00 |
| Euxyl 90/10 | 0.50 |
| Water | 64.50 |
| Total | 100.00 |

Oxidizing Phase

| Ingredient | % by weight |
|---|---|
| Hydrogen Peroxide (50%) | 7.75 |
| Pluronic F-127 | 2.50 |
| Water | 89.75 |
| Total | 100.00 |

Package in Rexam® Airspray Dual Foamer with Nitronic® Stainless Steel. This package dispenses equal volumes of each phase, as such, the concentration of hydrogen peroxide has been adjusted to compensate for differences in the density of the two phases. The mean rise in temperature upon mixing is ca. 57° F.

### Example 4

A lotion formula utilizing both oxidation-reduction and acid-base reactions to produce heat. This formula contains dimethicone, a skin protectant medicament.

Reducing Phase

| Ingredient | by weight |
|---|---|
| Sodium Sulfite | 8.00 |
| Dimethicone 500 cps | 2.00 |
| Span 80 | 5.00 |
| Stearyl Alcohol | 3.50 |
| Monafax 785 | 4.00 |
| Mineral Oil | 4.00 |
| Xanthan Gum | 1.00 |
| Germaben 2 | 0.25 |
| TEA, 99% | 7.65 |
| Water | 64.60 |
| Total | 100.00 |

Oxidizing Phase

| Ingredient | % by weight |
|---|---|
| Phosphoric Acid (85%) | 2.00 |
| Hydrogen Peroxide (50%) | 4.30 |
| Xanthan Gum | 1.00 |
| Water | 92.70 |
| Total | 100.00 |

The mean rise in temperature upon mixing is ca. 50° F.

1. An improved method of applying at least one dermally or transdermally physiologically active medicament to the skin of a mammal in need of same consisting essentially of
a) mixing a first dermally acceptable phase with a second dermally acceptable phase to provide a mixture, wherein at least one of said phases has an aqueous component,
wherein said medicament is present in either phase, and said first phase consists essentially of at least one component capable of producing an exothermic reaction of dermally acceptable magnitude when contacted with said second phase and wherein at least one of said phases has an aqueous component and
a) promptly applying said mixture to the skin of said subject.

2. The method of claim 1 wherein said first phase is a reducing phase and said second phase is an oxidizing phase.

3. The method of claim 1 wherein said first phase is a basic phase and said second phase is an acidic phase.

4. The method of claim 2 wherein said first phase additionally comprises a basic component and said second phase comprises an acidic component reactable with said basic component

5. The method of claim 2 wherein said first phase additionally comprises an acidic component and said second phase comprises a basic component reactable with said acidic component.

6. The method of claim 4 wherein the acidic phase is selected from the group consisting of sulfuric, hydrochloric, nitric, phosphoric, and water soluble organic acids with an equivalent weight of less than 100, the bases are selected from the group consisting of alkali and alkaline earth metal hydroxides, aqueous ammonia and amines, and the reducing agents are selected from the group consisting of alkali metal sulfites, thiosulphites and metabisulphites and the oxidizing agents is selected from dermally acceptable peroxides.

7. The method of claim 5 wherein the acidic phase is selected from the group consisting of sulfuric, hydrochloric, nitric, phosphoric, and water soluble organic acids with an equivalent weight of less than 100 selected from the group consisting of alkali and alkaline earth metal hydroxides, aqueous ammonia and amines, and the reducing agents are selected from the group consisting of alkali metal sulfites, thiosulphites and metabisulphites metabisulphites and the oxidizing agents is selected from dermally acceptable peroxides.

8. The method of claim 6 wherein the amount of reducing agent is between about 2-about 5 w/w %, the amount of acid is between about 0.5-about 5.0 w/w % both calculated on total weight of the phase and the amount of base is sufficient to result in a final mixture of pH between about 2-about 12.

9. The method of claim 6 wherein the amount of reducing agent is between about 2-about 5 w/w %, the amount of acid is between about 0.5-about 5.0 w/w % both calculated on total weight of the phase and the amount of base is sufficient to result in a final mixture of pH between about 2-about 12

10. A kit for applying at least one dermally or transdermally physiologically active medicament to the skin of a mammal in need of same consisting essentially of:
a first dermally acceptable phase and a separate second dermally acceptable phase mixable therewith,
wherein said medicament is present in either phase, and said first phase consists essentially of at least one component capable of producing an exothermic reaction of dermally acceptable magnitude when contacted with said second phase and wherein at least one of said phases has an aqueous component.

11. The kit of claim 6 wherein said first phase is a reducing phase and said second phase is an oxidizing phase.

12. The kit of claim 6 wherein said first phase is a basic phase and said second phase is an acidic phase.

13. The kit of claim 7 wherein said first phase additionally comprises a basic component and said second phase comprises an acidic component reactable with said basic component

14. The kit of claim 7 wherein said first phase additionally comprises an acidic component and said second phase comprises a basic component reactable with said acidic component.
